(19) United States
(12) Patent Application Publication
(10) Pub. No.: US 2010/0129872 A1
(43) Pub. Date: May 27, 2010

(54) SYSTEM AND METHOD FOR MOVEMENT AND POSITIONING OF REACTION MIXTURE DURING NUCLEIC ACID AMPLIFICATION

(75) Inventors: Philip Y. Paik, Chula Vista, CA (US); Michael R. Meyer, San Diego, CA (US)

Correspondence Address: James P. Broder;Roeder & Broder LLP, 9915 Mira Mesa Blvd. Suite 300, San Diego, CA, 92131, US

(73) Assignee: GHC Technologies, Inc.

(21) Appl. No.: 12/624,334

(22) Filed: Nov. 23, 2009

Related U.S. Application Data
(60) Provisional application No. 61/117,560, filed on Nov. 24, 2008.

(51) Int. Cl. C12P 19/34; C12M 1/00

(52) U.S. Cl. 435/91.1; 435/289.1

(57) ABSTRACT

A system for accurately positioning a reaction mixture during amplification of a nucleic acid includes a reaction vessel that contains the reaction mixture, a first heat exchanger, a second heat exchanger and a pump assembly. The reaction vessel can include .a first zone and a second zone. The first heat exchanger is positioned near the first zone, and the second heat exchanger positioned near the second zone. The first heat exchanger adjusts the temperature of the reaction mixture so the reaction mixture is at a first temperature when the reaction mixture is in the first zone. The second heat exchanger adjusts the temperature of the reaction mixture so the reaction mixture is at a second temperature. The pump assembly adjusts the pressure within the reaction vessel to selectively position the reaction mixture relative to the first zone and the second zone during amplification. The system can include a sensor that monitors the position of the reaction mixture within the reaction vessel. The system can also include a second pump that cooperates with the pump assembly to adjust the position of the reaction mixture within the reaction vessel.

### REFERENCE TO RELATED APPLICATION

This Application claims domestic priority on U.S. Provisional Application Ser. No. 61/117,560, filed on Nov. 24, 2008. The contents of U.S. Provisional Application Ser. No. 61/117,560 are incorporated herein by reference to the extent permitted.

### BACKGROUND

Deoxyribonucleic acid (DNA) may be amplified by thermally cycling a specially constituted liquid reaction mixture according to protocol such as a polymerase chain reaction (PCR) protocol which includes several incubations at different temperatures. The reaction mixture is comprised of various components such as a DNA template sought to be amplified (the target) and at least two oligonucleotide primers selected in a predetermined way so as to be complementary to a portion of the target DNA. The reaction mixture can also include various buffers, enzymes, and deoxyribonucleotide triphosphates, such as dATP, dCTP, dGTP, and dTTP. The target molecule is denatured into two complementary single strands. In PCR, the primers then anneal to the strands and nucleoside monophosphate residues are then linked to the primers in the presence of an enzyme such as a DNA polymerase to create a primer extension product. After primer extension, twice as many target molecules exist. This process is repeated, each time approximately doubling the amount of target DNA molecules present. The result is an exponential increase in the concentration of the target DNA, known as “amplification”.

PCR has proven to be a success for genetic analysis, largely because it is fairly simple and quite versatile, and requires relatively low cost instrumentation. One key to this success is the concept of thermal cycling: alternating steps of denaturing of the DNA, annealing short primers to the resulting single strands, and extending those primers to make new copies of the double stranded DNA. PCR has been performed in disposable reaction tubes such as small, plastic microcentrifuge tubes or test tubes which are placed in an instrument containing a thermally controlled heat exchanger. The heat exchanger in these instruments can be a metal block; however, hot air ovens and water baths also have been used. The temperature of the reaction mixture in the reaction tubes is changed in a cyclical fashion to cause denaturation, annealing and extension reactions to occur in the mixture. Two or more separate incubation temperatures can be used, including approximately 94° C. for denaturation, and approximately 50°-65° C. for annealing and extension incubation. As used herein, a system that moves the reaction mixture between two different temperature heat exchangers is referred to as “shuttle PCR”.

Specificity of primer-based amplification depends on the specificity of primer hybridization. Ideally, under the elevated temperatures used in a typical amplification, the primers should theoretically hybridize only to the target sequence. However, there is a relatively narrow range of conditions (i.e. temperature, concentrations of ions and denaturing agents) for specific annealing of an oligonucleotide primer to its complementary target. When conditions fall outside this range, non-specific annealing of primers either to themselves or to other sequences in the reaction may occur and result in amplification of nonspecific product. Annealing of primers to themselves or to other primers in the reaction is referred to herein as “primer-dimer.” Non-specific amplification of primer-dimer products is particularly problematic in PCR since primers are typically present at high concentrations in the PCR reaction. The high concentration of primer facilitates weak interactions between non-complementary oligonucleotides, resulting in primer-dimer formation, particularly at reduced annealing temperatures. These interactions result in the amplification of undesired non-specific products during PCR which can compete with amplification of the desired specific target sequence and can significantly decrease the efficiency of the amplification of the desired product.

Because of the requirement of a relatively narrow range of conditions for specific annealing of an oligonucleotide primer to its complementary target, the occurrence of primer-dimer has been extremely difficult to inhibit or eliminate. Primer sequences are designed specifically to match a certain target sequence and to specifically anneal to a complementary sequence at a specific temperature. However, at a temperature that is too high, annealing of primers to the target sequence does not occur or occurs inefficiently, resulting in reduced amplification efficiency of the target sequence. At a temperature that is too low, non-specific annealing of primers, either to non-target sequences in the template DNA or to other primers in the reaction occurs.

Multiplex PCR is a form of PCR where multiple primer pairs are present in a single reaction, each targeting a different sequence of DNA and amplifying a different target. In multiplex PCR, greater opportunities for primer-dimer and non-specific amplification are present due to the increased abundance of different sequences at high concentrations and the increased potential for non-specific interactions among non-complementary sequences. Accurately controlling incubation temperatures during PCR thermal cycling becomes even more critical in these applications since even a small number of non-specific annealing events can result in the generation of a non-target amplicon that becomes available for amplification during subsequent rounds of PCR, resulting in exponential amplification of the non-specific product, often at the expense of amplification efficiency of the target sequence.

Additionally, under elevated temperatures required for PCR, evaporation of the fluid sample can occur. During shuttle PCR, evaporation that occurs in the capillary will condense in the cooler regions of the capillary that are outside of the temperature controlled zones. The condensate that forms outside of the controlled temperature zones can mix with the residual PCR sample coating the capillary and result in annealing and extension products, but at a much lower stringency than intended, resulting in increased chance for primer-dimer and other non-specific amplification products. These non-specific products can mix with the primary fluid sample every time it shuttles back and forth between the two zones. Any mixing with the condensate will introduce one or more non-specific amplicons into the PCR reaction, which can compete with amplification of the specific target.

Evaporative loss of the liquid PCR sample can also result in a concentration effect of the salts, nucleotides, primers, etc. in the reaction. This evaporative loss can lead to an imbalance of components whose proper concentrations relative to one another can be critical for achieving sensitivity and specificity of the amplification reaction. Improper concentrations of the reaction components can result in loss of amplification efficiency as well as increased formation of non-specific amplification products.

While the fluid sample is positioned in a controlled temperature zone in the capillary, thermocapillary forces can exert a strong influence on the liquid slug, often resulting in migration of the sample out of the temperature controlled zone, either towards a cooler or hotter zone, depending on whether the capillary is hydrophobic or hydrophilic (more accurately, depending on whether the contact angle is greater than 90 degrees or less than 90 degrees). Portions of the liquid slug that fall outside of the temperature controlled zones will likely undergo annealing and extension at suboptimal temperatures, resulting in increased amplification of non-specific products.

Various conventional methods for controlling and accurately positioning reaction mixture during shuttle PCR have drawbacks. Oil slugs positioned on either side of the liquid PCR sample are often used to contain the sample within the heated zones and to minimize evaporation; however, oil often needs to be removed for downstream analysis of the product, adding complexity to the process, especially in an automated system. Oil may also introduce a contaminant or an inhibitor to the sample as well as decreasing the rate of thermal equilibrium of the sample due to added thermal mass and heat capacity of the oil layer, thus increasing the total cycle time.

### SUMMARY

The present invention is directed toward a system for accurately positioning a reaction mixture during amplification of a nucleic acid. In one embodiment, the system includes a reaction vessel that contains the reaction mixture, a first heat exchanger and a pump assembly. The reaction vessel can include one or more zones. In one embodiment, the reaction vessel includes a first zone and a second zone. The first heat exchanger is positioned near the first zone. The first heat exchanger adjusts the temperature of the reaction mixture so the reaction mixture is at a first temperature when the reaction mixture is in the first zone. The pump assembly adjusts the pressure within the reaction vessel to a level that is greater than atmospheric pressure. The pump assembly selectively positions the reaction mixture within the first zone during amplification of the nucleic acid. In one embodiment, the reaction vessel is a capillary tube.

In certain embodiments, the system also includes a second heat exchanger positioned near the second zone. The second heat exchanger adjusts the temperature of the reaction mixture so the reaction mixture is at a second temperature that is different than the first temperature. In one embodiment, the pump assembly selectively positions the reaction mixture in the second zone by adjusting the pressure within the reaction vessel. In some embodiments, the pump assembly cyclically positions the reaction mixture between the first zone and the second zone during amplification of the nucleic acid. In accordance with one embodiment, the first heat exchanger adjusts the temperature of the reaction mixture to within a range of approximately 90-98° C. Additionally, or in the alternative, the second heat exchanger adjusts the temperature of the reaction mixture to within a range of approximately 50-65° C.

In one embodiment, the system also includes a sensor that monitors the position of the reaction mixture within the reaction vessel. The system can also include an input channel including an input channel pinch valve that selectively opens and closes to control movement of the reaction mixture relative to the reaction vessel. In certain embodiments, the system includes an output channel including and output channel pinch valve that selectively opens and closes. In this embodiment, the output channel pinch valve cooperates with the input channel pinch valve to control movement of the reaction mixture relative to the reaction vessel. In one embodiment, the output channel includes an air reservoir that regulates the pressure within the reaction vessel.

In another embodiment, the system also includes an input channel including a second pump that cooperates with the pump assembly to adjust the position of the reaction mixture within the reaction vessel.

The present invention is also direction toward a method for positioning a reaction mixture within a reaction vessel during amplification of a nucleic acid.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic diagram illustrating one embodiment of a system for movement and positioning of a reaction mixture during nucleic acid amplification having features of the present invention;

FIG. 2 is a flow chart outlining one embodiment of a method for positioning a reaction mixture during amplification of a nucleic acid;

FIG. 3 is a simplified schematic diagram illustrating another embodiment of the system for movement and positioning of the reaction mixture during nucleic acid amplification;

FIG. 4A is a top perspective view of one embodiment of a shuttle PCR module system for movement and positioning of the reaction mixture during nucleic acid amplification;

FIG. 4B is a bottom perspective view of the shuttle PCR module system illustrated in FIG. 4A;

FIG. 5 is a flow chart outlining another embodiment of a method for positioning the reaction mixture during amplification of a nucleic acid; and

FIG. 6 is a simplified schematic diagram illustrating yet another embodiment of the shuttle PCR module system for movement and positioning of the reaction mixture during nucleic acid amplification.

### DESCRIPTION

As an overview, various embodiments of the present invention utilize pressurization to control evaporation of the fluid sample, to prevent sample volume loss and/or to prevent accumulation of condensate outside of the temperature controlled zone. Alternatively, or in addition, the use of pressurization can increase the accuracy of the positioning of the sample within one or more temperature-controlled zones, which can consequently decrease the incidence of primer-dimer.

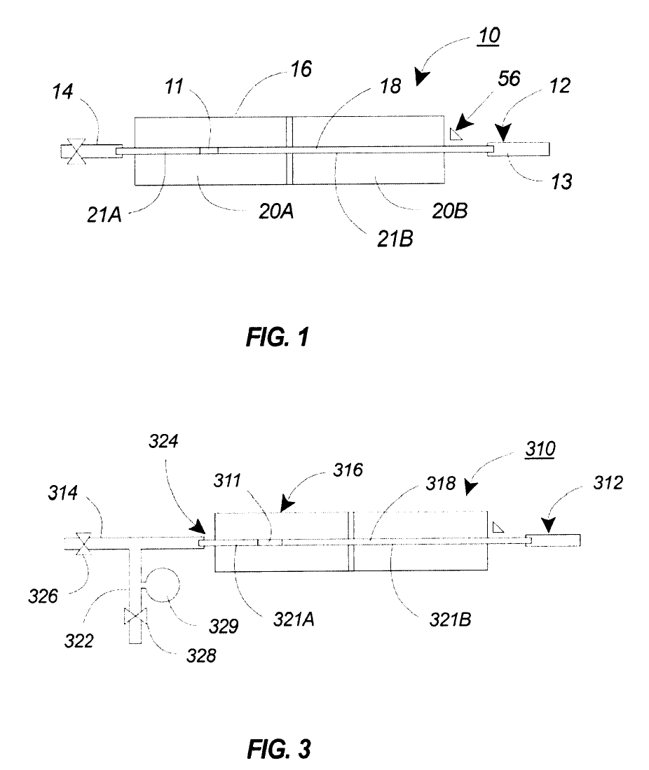

FIG. 1 is a simplified schematic diagram illustrating one embodiment of a shuttle PCR module system 10 (also sometimes referred to herein as “system”) for movement and positioning of a reaction mixture 11 (also sometimes referred to herein as a “sample”) during nucleic acid amplification. In the embodiment illustrated in FIG. 1, the system 10 includes a pump assembly 12, an input channel 14 and a shuttle PCR module 16 (also sometimes referred to herein as “module”). The pump assembly 12 moves the sample 11 to and from the module 16 as well as within the module 16. The pump assembly 12 can include one or more pumps 13 (only a “first pump” 13 is illustrated in FIG. 1). In one embodiment, the input channel 14 can include a sample retainer (not illustrated in FIG. 1) and/or a conduit for transferring the sample 11 from the sample retainer to the module 16.

In the embodiment illustrated in FIG. 1, the module 16 includes one or more reaction vessels 18 (e.g., capillary tubes or the like), and two or more heat exchangers including a first heat exchanger 20A positioned at or near an annealing zone 21A (also sometimes referred to herein as one of a “first zone” or a “second zone”), a second heat exchanger 20B positioned at or near a denature zone 21B (also sometimes referred to herein as one of a “first zone” or a “second zone”), and a sensor 56. The first heat exchanger 20A is initially set to an annealing temperature, and the second heat exchanger 20B is initially set to a denaturing temperature. The specific annealing and/or denature temperatures can vary depending upon the design requirements of the system 10 and the specific nucleic acid sought to be amplified. The sensor 56 can include any suitable type of sensor, such as an optical sensor as one non-exclusive example.

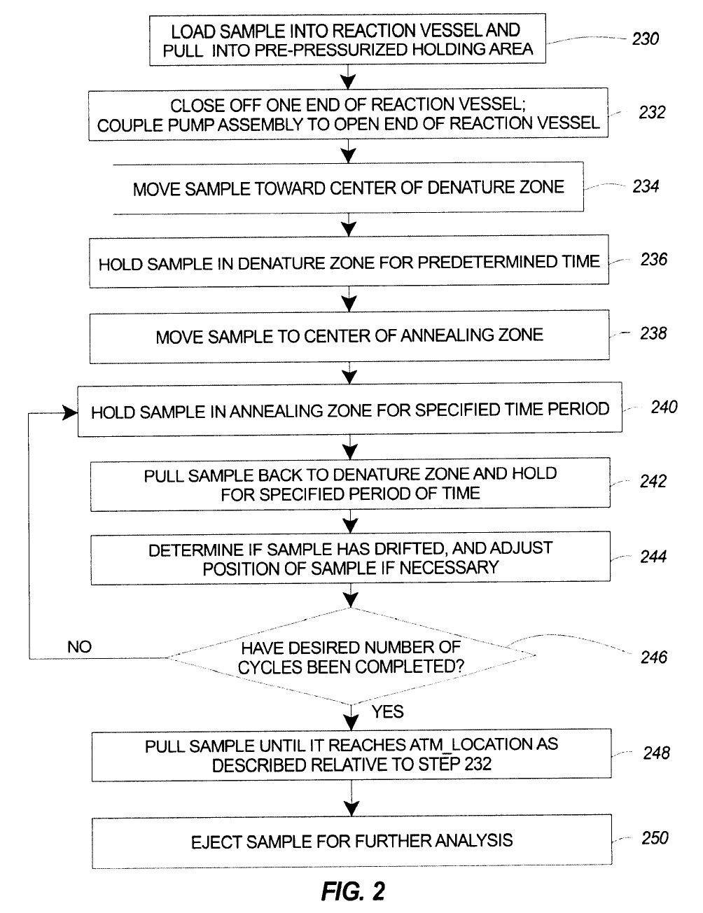

FIG. 2 is a flow chart outlining one method for positioning the reaction mixture during amplification of the nucleic acid, in accordance with one embodiment of the invention. The example below is provided for ease of discussion only, and it is recognized that one or more of the steps below can be omitted, other steps can be added, and/or the specific parameters in one or more of the steps can be altered depending upon the specific nucleic acid sought to be amplified.

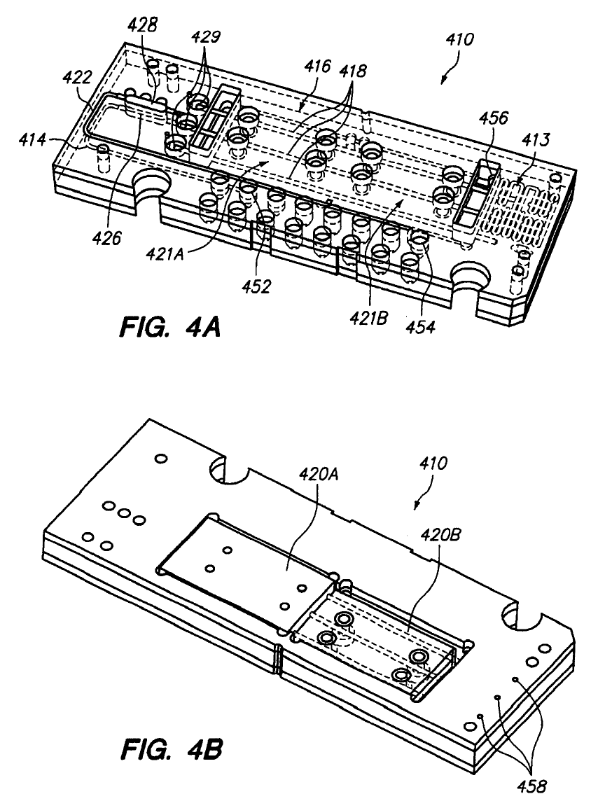

At step 230, a sample can be loaded into a capillary tube or another suitable vessel (by any suitable means and at any suitable end of the capillary tube or vessel) at atmospheric pressure, and pulled into a pre-pressurized holding area 413 (illustrated in FIG. 4A) that has been pressurized using a pump assembly. A sensor (such as an optical sensor or another suitable sensor) can be used to determine when the sample has been positioned in the holding area.

At step 232, a pinch valve or another suitable structure can be actuated to close off one end of the capillary tube, leaving an opposing end of the capillary tube open. The pump assembly is coupled to interface with the open end of the capillary tube. At the position of the pump assembly, the pressure is approximately equal to atmospheric pressure (ATM_LOCATION). In one embodiment, the pump assembly includes one pump that pressurizes the holding area and also pushes the sample. Alternatively, the pump assembly can include a first pump that pressurizes the holding area, and a second pump that pushes the sample.

At step 234, the pump pushes the sample toward the center of the denature zone, and can use the sensor to assist in determining the location of the sample.

At step 236, the sample is now allowed to sit in the denature zone for a specified hot start activation time, which is the time required to activate an enzyme, such as a Hot-Start Taq Polymerase enzyme, in one non-exclusive embodiment. In one non-exclusive embodiment, the hot start activation time can be approximately 30 seconds. However, it is recognized that the hot-start activation time can be greater or less than 30 seconds. In one embodiment, the denature zone is maintained within the range of 90-98° C. In another embodiment, the denature zone is maintained at approximately 94° C.

At step 238, the sample is then pushed by X units toward the center of the annealing zone. The amount that is needed to push can be determined empirically in a prior experiment, and can remain the same across all subsequent experiments given that the geometries of the capillary and tube lengths are consistent. Alternatively, the amount that is needed to push can be determined by another suitable method.

At step 240, the sample is then held in the annealing zone for a specified annealing time. In one embodiment, the annealing time can be approximately 8 seconds. However, it is recognized that the annealing time can be greater or less than 8 seconds. In one embodiment, the annealing zone is maintained within the range of 50-65° C. In another embodiment, the denature zone is maintained at approximately 63° C. The sample has now completed one cycle of PCR.

At step 242, the sample is pulled back to the denature zone by pulling X units, and held for a specified denaturation time. The number of units comprising X can vary depending upon various design parameters of the system. In one embodiment, the denaturation time can be approximately 1 second. However, it is recognized that the denaturation time can be greater or les s than 1 second.

At step 244, the sensor can also be polled at this time to determine whether the sample has drifted. Drifting can be caused by the gradual heating of the air inside the capillary tube, which gets mixed as the sample is pushed back and forth. In one embodiment, if the sample is determined to have drifted, an extra Y units can be pushed by the pump to account for the drift. The number of units comprising Y can vary.

At step 246, steps 240-244 can be repeated until the desired number of cycles are completed.

At step 248, the sample is pulled until it reaches ATM_LOCATION which was determined in step 232.

At step 250, the pinch valve is opened, and the sample is allowed to be ejected for further analysis, if determined to be necessary.

In an alternative embodiment, the module 16 can include only one heat exchanger (similar to the first heat exchanger 20A or the second heat exchanger 20B). In one such embodiment, the heat exchanger can fluctuate between an annealing temperature and a denaturing temperature, while the reaction mixture is positioned within the reaction vessel adjacent to the heat exchanger by the system and methods provided herein during amplication of the nucleic acid. This temperature fluctuation can occur in a cyclical manner as many times as required to achieve the desired level of amplification.

In yet another embodiment, the module 16 can include only one heat exchanger (similar to the first heat exchanger 20A or the second heat exchanger 20B). In this embodiment, the reaction mixture is moved by the pump assembly from a first location adjacent to the heat exchanger to a different location that is away from the heat exchanger. For example, the heat exchanger can be set at a denaturing temperature. Once the reaction mixture is moved away from the denaturing temperature to a lower temperature within the reaction vessel, annealing can occur. In this embodiment, the reaction mixture can be cycled back and forth between the two different temperature areas within the reaction vessel in order to achieve the required number of PCR cycles.

### Shuttle PCR Module Interface

FIG. 3 illustrates one embodiment of how a shuttle PCR module 316 as described herein can be interfaced. For example, in one embodiment, the module 316 can be in a cartridge format as part of a shuttle PCR module system 310 that allows for sample 311 preparation and post-PCR analysis. In one embodiment, the system 310 includes a pump assembly 312, an input channel 314, the module 316 and an output channel 322. The input channel 314 and the output channel 322 can be connected to an input side 324 of the reaction vessel 318 of the module 316. The input channel 314 can include an input channel pinch valve 326 in order to open and/or close access to the input channel 314. Somewhat similarly, the output channel 322 can include an output channel pinch valve 328 that opens and/or closes access to the output channel 322.

Additionally, in one embodiment, the output channel 322 can also include air reservoir 329. The air reservoir 329 regulates the final pressure values within the module 316. In one such embodiment, the pressures can equal roughly 3.5 psi when the sample 311 is in the annealing zone 321A, and roughly 1.0 psi when the sample 311 is in the denature zone 321B. Making the air reservoir 329 smaller would increase both pressures, while making the air reservoir 329 larger would decrease both pressures. In certain embodiments, the specific pressures in each of the annealing zone 321A and the denature zone 321B can vary from the example provided above, provided that the pressures are greater than atmospheric pressure.

In another embodiment, the entire system 310 can be pressurized. For example, not only is the interior of the reaction vessel 318 pressurized, but the entire system 310 can be included in the contents of a container (not shown), the interior of which is pressurized to a level that is greater than atmospheric pressure.

FIG. 4A is a top perspective view of one embodiment of a shuttle PCR module system 410 for movement and positioning of a reaction mixture during nucleic acid amplification. In this embodiment, the system 410 includes a pre-pressurized holding area 413, an input channel 414, a shuttle PCR module 416, one or more reaction vessels 418, a first heat exchanger 420A (illustrated in FIG. 4B) positioned at or adjacent to an annealing zone 421A, and a second heat exchanger 420B (illustrated in FIG. 4B) positioned at or adjacent to a denature zone 421B, an output channel 422, an input channel pinch valve 426, an output channel pinch valve 428, one or more air reservoirs 429, one or more input wells 452, one or more output wells 454 and a sensor 456. In the embodiment illustrated in FIG. 4A, the input channel 414 is connected to the one or more input wells 452, and the output channel 422 can be connected to the one or more output wells 454.

FIG. 4B is a bottom perspective view of the shuttle PCR module system 410 illustrated in FIG. 4A. In the embodiment illustrated in FIG. 4B, the system 410 includes one or more pump interfaces 458 (three pump interfaces 458 are illustrated in FIG. 4B). The pump interfaces 458 interface with the pump assembly (not illustrated in FIG. 4B), such as the pump assembly 12 illustrated in FIG. 1, to allow pressurization of the pre-pressurized holding area 413 (illustrated in FIG. 4A).

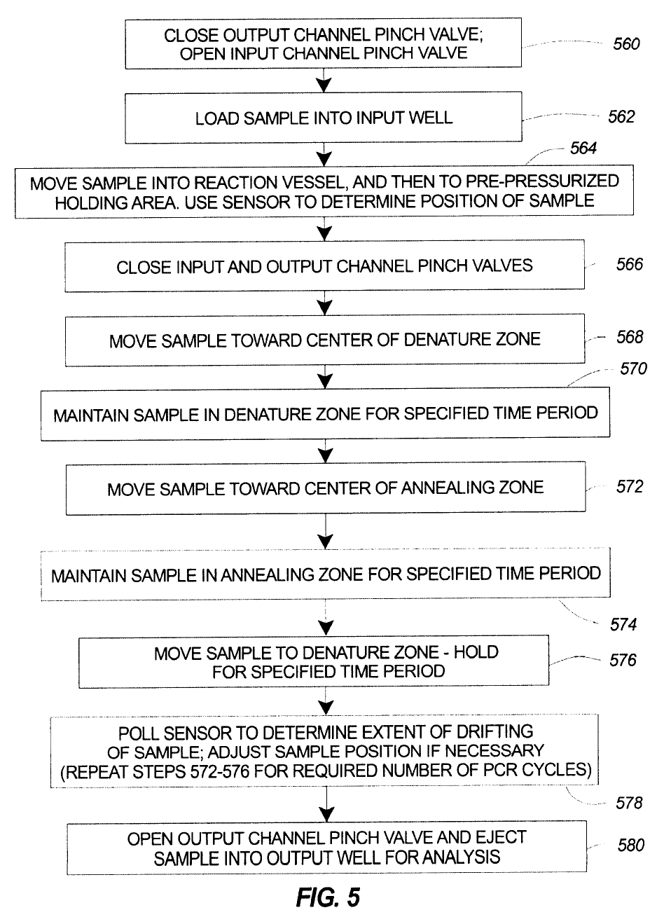

FIG. 5 is a flow chart outlining a method for positioning the reaction mixture during amplification of a nucleic acid, in accordance with another embodiment of the invention. The following steps represent one non-exclusive example for loading and ejecting the sample into the shuttle PCR module.

At step 560, the output channel pinch valve is closed, while the input channel valve is opened.

At step 562, the sample is loaded into the input well that is connected to the input channel.

At step 564, the sample can be loaded into the reaction vessel (such as a capillary tube) at atmospheric pressure. The sample is then pulled into the pre-pressurized holding area. The sensor can be used to determine when the sample has been positioned into the holding area.

At step 566, the input and output channel pinch valves are actuated to close. In one embodiment, at the position of the pump assembly, the pressure is at or around atmospheric pressure (ATM_LOCATION).

At step 568, the pump assembly moves the sample toward the center of the denature zone. The sensor can be used to assist in determining the location of the sample.

At step 570, the sample is now allowed to remain in the denature zone for a specified hot start activation time, which is the time required to activate an enzyme, such as a Hot-Start Taq Polymerase enzyme, in one non-exclusive embodiment. In one non-exclusive embodiment, the hot start activation time can be approximately 30 seconds. However, it is recognized that the hot-start activation time can be greater or less than 30 seconds.

At step 572, the sample is then pushed by X units toward the center of the annealing zone. The amount that is needed to push can be determined empirically in a prior experiment, and can remain the same across all subsequent experiments given that the geometries of the capillary and tube lengths are consistent. Alternatively, the amount that is needed to push can be determined by another suitable method.

At step 574, the sample is then held in the annealing zone for a specified annealing time. In one embodiment, the annealing time can be approximately 8 seconds. However, it is recognized that the annealing time can be greater or less than 8 seconds. The sample has now completed one cycle of PCR.

At step 576, the sample is pulled back to the denature zone by pulling X units, and held for a specified denaturation time. The number of units comprising X can vary depending upon various design parameters of the system. In one embodiment, the denaturation time can be approximately 1 second. However, it is recognized that the denaturation time can be greater or less than 1 second.

At step 578, the sensor can also be polled to determine whether the sample has drifted. Drifting can be caused by the gradual heating of the air inside the capillary tube, which gets mixed as the sample is pushed back and forth. In one embodiment, if the sample is determined to have drifted, an extra Y units can be pushed by the pump to account for the drift. The number of units comprising Y can vary.

At step 580, the output channel pinch valve is opened, the input channel pinch valve remains closed, and the sample is ejected into the output well for further analysis, if necessary.

### Thermal & Pressure Interfaces

In one embodiment, the heat exchangers illustrated and described herein can consist of on-cartridge aluminum blocks containing grooves that mate with, surround or at least partially encircle the capillary tube. In certain embodiments, one or more heaters can either be applied directly onto the blocks, or they can be implemented on separate blocks to which they could mate. The heaters can either be resistive heating elements, peltier elements, or other suitable devices that can generate heat. If separate heating blocks are used to interface with the on-cartridge blocks, thermal resistance can be reduced by either polishing both blocks, using a thermal interface material such as thermal paste, or by other suitable means. Similarly, the thermal resistance between the on-cartridge blocks can be reduced in the same fashion.

The reduction of thermal resistances can be implemented to assist in maintaining accurate and precise temperatures. In one embodiment, temperatures can be regulated by using a proportional-integral-derivative (PID) controller, which can be implemented on the block to which the heater is connected. Alternatively, methods for directly heating the reaction vessel can also be used, i.e. coating the capillary tube with a thin layer of indium tin oxide or some other metal or metal alloy, and applying a current across it, as alternative non-exclusive examples.

The interface by which the pump assembly can connect to the module can include fluidic connectors or any other suitable connector. In one embodiment, the interface can include the use of rubber, compressible O-rings which compress against the pump assembly interface holes illustrated in FIG. 4B. The amount of pressure the O-rings can withstand can be proportional to the compression force applied to the O-rings.

In one non-exclusive alternative embodiment, the pressure can vary depending upon the location of the sample. In certain such embodiments, one end of the reaction vessel is sealed off. The result is that the pressure inside the reaction vessel at the denature zone will be lower than the pressure inside the reaction vessel at the annealing zone. This design could result in an increased chance of evaporation and condensation at the denaturation end. Consequently, in one embodiment, the denature zone and the annealing zone are reversed. Because evaporation is more likely to occur due to the higher temperatures at the denature zone, having a higher pressure at that location would mitigate or otherwise reduce evaporation and/or condensation.

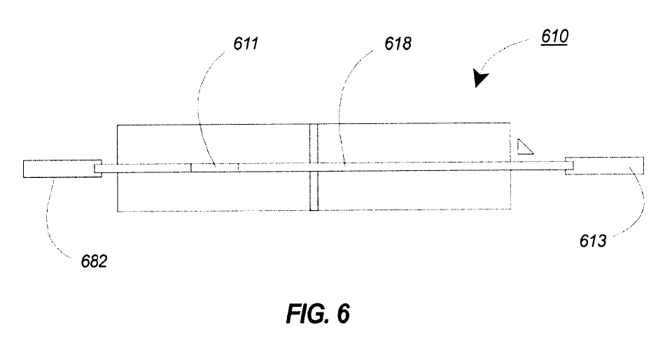

FIG. 6 is a simplified schematic diagram illustrating yet another embodiment of the shuttle PCR module system 610 for movement and positioning of the reaction mixture 611 during nucleic acid amplification. In this embodiment, rather than having one end of the reaction vessel 618 sealed off, the input channel pinch valve 326 (illustrated in FIG. 3) can be replaced with an additional pump 682 (also referred to herein as a “second pump”), as illustrated in FIG. 6. This would allow a substantially similar or equal pressure to be maintained during the PCR cycling process by allowing both pumps 613, 682 to push against the sample 611 once the sample 611 is loaded, in order to pressurize the sample 611. The pumps 613, 682 cooperate with one another and can consequently move in unison (one pump pushing the same amount the other pump is pulling), to move the sample 611 back and forth while maintaining the same pressure in the reaction vessel 618.

In this embodiment, the depressurization step can be simplified by eliminating the need for the pre-pressurization holding area 413 (illustrated in FIG. 4A), since pressure can be equalized from both sides of the sample 611 without any substantial sample drift.

While the systems and methods as shown and disclosed herein are fully capable of obtaining the objects and providing the advantages herein before stated, it is to be understood that they are merely illustrative of the presently preferred embodiments of the invention and that no limitations are intended to the details of the methods, processes, construction or design herein shown and described.

What is claimed is:

1. A system for accurately Positioning a reaction mixture during amplification of a nucleic acid, the system comprising:
a reaction vessel that contains the reaction mixture, the reaction vessel including a first zone and a second zone;
a first heat exchanger positioned near the first zone, the first heat exchanger adjusting the temperature of the reaction mixture so the reaction mixture is at a first temperature when the reaction mixture is in the first zone; and
a pump assembly that adjusts the pressure within the reaction vessel to a level that is greater than atmospheric pressure, the pump assembly selectively positioning the reaction mixture within the first zone during amplification of the nucleic acid.

2. The system of claim 1 wherein the reaction vessel is a capillary tube.

3. The system of claim 1 further comprising a second heat exchanger positioned near the second zone, the second heat exchanger adjusting the temperature of the reaction mixture so the reaction mixture is at a second temperature that is different than the first temperature when the reaction mixture is in the second zone.

4. The system of claim 3 wherein the pump assembly selectively positions the reaction mixture in the second zone by adjusting the pressure within the reaction vessel.

5. The system of claim 4 wherein the pump assembly cyclically positions the reaction mixture between the first zone and the second zone during amplification of the nucleic acid.

6. The system of claim 5 wherein the first heat exchanger adjusts the temperature of the reaction mixture to within a range of approximately 90-98° C.

7. The system of claim 6 wherein the second heat exchanger adjusts the temperature of the reaction mixture to within a range of approximately 50-65° C.

8. The system of claim 1 further comprising a sensor that monitors the position of the reaction mixture within the reaction vessel.

9. The system of claim 1 further comprising an input channel including an input channel pinch valve that selectively opens and closes to control movement of the reaction mixture relative to the reaction vessel.

10. The system of claim 9 further comprising an output channel including and output channel pinch valve that selectively opens and closes, the output channel pinch valve cooperating with the input channel pinch valve to control movement of the reaction mixture relative to the reaction vessel.

11. The system of claim 10 wherein the output channel includes an air reservoir that regulates the pressure within the reaction vessel.

12. The system of claim 1 further comprising an input channel including a second pump that cooperates with the pump assembly to adjust the position of the reaction mixture within the reaction vessel.

13. A system for accurately positioning a reaction mixture during amplification of a nucleic acid, the system comprising:
a reaction vessel that contains the reaction mixture, the reaction vessel including a first zone and a second zone;
a first heat exchanger positioned near the first zone, the first heat exchanger adjusting the temperature of the reaction mixture so the reaction mixture is at a first temperature when the reaction mixture is in the first zone;
a second heat exchanger positioned near the second zone, the second heat exchanger adjusting the temperature of the reaction mixture so the reaction mixture is at a second temperature that is different than the first temperature when the reaction mixture is in the second zone; and
a pump assembly that adjusts the pressure within the reaction vessel to move the reaction mixture relative to the first heat exchanger and the second heat exchanger during amplification of the nucleic acid.

14. The system of claim 13 wherein the pump assembly cyclically positions the reaction mixture between the first zone and the second zone during amplification of the nucleic acid.

15. The system of claim 14 wherein the first heat exchanger adjusts the temperature of the reaction mixture to within a range of approximately 90-98° C.

16. The system of claim 15 wherein the second heat exchanger adjusts the temperature of the reaction mixture to within a range of approximately 50-65° C.

17. The system of claim 13 further comprising a sensor that monitors the position of the reaction mixture within the reaction vessel.

18. The system of claim 13 further comprising an input channel including an input channel pinch valve that selectively opens and closes to control movement of the reaction mixture relative to the reaction vessel.

19. The system of claim 18 further comprising an output channel including and output channel pinch valve that selectively opens and closes, the output channel pinch valve cooperating with the input channel pinch valve to control movement of the reaction mixture relative to the reaction vessel.

20. The system of claim 19 wherein the output channel includes an air reservoir that regulates the pressure within the reaction vessel.

21. A method for positioning a reaction mixture within a reaction vessel during amplification of a nucleic acid, the method comprising the steps of:
adjusting the temperature of a first zone of the reaction vessel with a first heat exchanger;
adjusting the temperature of a second zone of the reaction vessel with a second heat exchanger so that the temperature within the second zone is different than the temperature of the first zone; and
adjusting the pressure within the reaction vessel with a pump assembly to position the reaction mixture within the reaction vessel relative to the first zone and the second zone.

22. The method of claim 21 wherein the step of adjusting the pressure includes the pump assembly cyclically positioning the reaction mixture between the first zone and the second zone.

23. The method of claim 22 wherein the step of adjusting the temperature of the first heat exchanger includes adjusting the temperature of the reaction vessel to within a range of approximately 90-98° C.

24. The method of claim 23 wherein the step of adjusting the temperature of the second heat exchanger includes adjusting the temperature of the reaction vessel to within a range of approximately 50-65° C.

25. The method of claim 21 further comprising the step of monitoring the position of the reaction mixture within the reaction vessel with a sensor.

26. The method of claim 21 further comprising the step of controlling movement of the reaction mixture within the reaction vessel by selectively opening and closing an input channel pinch valve of an input channel.

27. The method of claim 26 further comprising the step of controlling movement of the reaction mixture relative to the reaction vessel by selectively opening and closing an output channel pinch valve of an output channel in cooperation with the input channel pinch valve.

28. The method of claim 27 further comprising the step of regulating the pressure within the reaction vessel with an air reservoir of the output channel.

29. The method of claim 21 wherein the step of adjusting the pressure includes adjusting the position of the reaction mixture using a second pump of the input channel in cooperation with the pump assembly.
